(19) United States
(12) Patent Application Publication
(10) Pub. No.: US 2010/0305303 A1
(43) Pub. Date: Dec. 2, 2010

(54) INHIBITING AGENT FOR INHIBITION OF ANGIOGENESIS, A METHOD FOR PREPARING THE AGENT, A METHOD FOR MODIFYING THE AGENT AND ITS USE FOR MANUFACTURING A MEDICAMENT FOR TREATING TUMOR

(76) Inventor: Hanmei Xu, Nanjing (CN)

Correspondence Address: SCHMEISER, OLSEN & WATTS, 22 CENTURY HILL DRIVE, SUITE 302, LATHAM, NY, 12110, US

(21) Appl. No.: 12/665,746

(22) PCT Filed: Feb. 13, 2008
(86) PCT No.: PCT/CN08/00346

§ 371 Date: Jun. 10, 2010

(30) Foreign Application Priority Data
Jun. 22, 2007 (CN) 200710024565.3

(51) Int. Cl. C07K 7/08; C07K 9/00

(52) U.S. Cl. 530/322; 530/326; 530/327

(57) ABSTRACT

a highly efficient antiangiogenesis agent, which is a polypeptide for inhibition of angiogenesis Ile-Val-Arg-Arg-Ala-Asp-Arg-Ala-Ala-Val-Pro, connected with a polypeptide containing Arg-Gly-Asp on its one end or two ends is provided. The inhibiting agent can be synthesized or gene engineered. It also relates to a physiochemical method for modifying the antiangiogenesis agent. Polypeptides with weight percentage of 1-70% preferably about 20-50% are mixed with 20%-95% polyethylene glycol, or heparin, or dextran, or polyvinylpyrrolidone, or polyethylene glycol-poly-amino acid copolymer, or palmitic acid or poly-sialic acid or liposomes solutions; preferably about 50-93% of the above modified substances are fully mixed even and shaken at a shaker at 4° C.-40° C., preferably 25° C.-37° C. for more than 10 min, and the modified substances are separated through appropriate methods. Furthermore, it still relates to the use of the above polypeptides and the polypeptide modified substances for manufacturing medicaments for treating human solid tumors.

### FIELD OF THE TECHNOLOGY

The following relates to biomedical technology field or protein polypeptide drug field, particularly relates to an antiangiogenesis agent, the production and modification method thereof and the use for manufacturing medicaments for treating tumors.

### BACKGROUND

Tumor angiogenesis inhibitor is new type of drug focused on in the treatment of tumors in recent years. Studies in this field have been made and it is expected to be a new type of promising drug for treatment of tumors in the future. Algureza proposed the concept of tumor angiogenesis in 1947 and he pointed out that, one of the important features of tumor growth is to form from the new capillary endothelial cells of the host. In 1971, Folkman proposed the hypothesis that, the tumor growth and metastasis depend on angiogenesis and believed that during the primary solid tumor period, tumor angiogenesis factors may be secreted to stimulate the host capillary proliferation. Tumor angiogenesis can not only provide tumors with the needed nutrients and oxygen and to remove the metabolites, but also can form the path of distant metastasis (Folkman, J., J. Natl. Cancer Inst. 1990; 82:4-6). Therefore, blocking angiogenesis could become a means of preventing tumor growth and metastasis, and thus triggering the extensive study on the angiogenesis molecules and anti-angiogenesis molecules. Among these angiogenesis inhibitors, angiostatin and endostatin are most striking in particular, and both of which have been listed for the clinical trials in the United States, although these angiogenesis inhibitors present very attractive prospect, its flaws are also very clear: to date, the active targets of the angiogenesis drugs, such as endostatin and angiostatin, etc, are not clear, have no clear specificity and selectivity and limited effect, causing very high consumption in the test. In the animal model test of a mouse, the dose of angiostatin can be up to hundreds of mg/kg body weight, and the dose of endostatin can be up to dozens of mg/kg body weight. When the angiogenesis inhibitors are used in human body, the dose should be at least a few grams/person. Such high drug use dose will surely enhance the side effect of such type of drugs in the future, causing increased drug quality control difficulty, increased production scale and production costs and high drug prices.

Thus, a good anti-angiogenic drug shall have selectivity on the tagged molecules of the neovascularization to achieve a guiding role in angiogenesis and enhance the inhibitive role of the drugs on the angiogenesis from the overall aspect: to use a very low dose of drugs to achieve high effect of inhibition of angiogenesis.

Integrin is a transmembrane protein heterodimer composed of α and β subunits. The study shows that, the integrin on the tumor cell surface is the key for tumor metastasis, which controls the cell 1 migration, differentiation and proliferation through connecting the intracellular cytoskeleton and extracellular matrix protein interaction (Schoenwaelder S M, etc., Curr Opin Cell Biol, 1999; 274-286). The majority of more than 20 types of integrins can identify the extracellular matrix ligand containing the RGD (arginine-glycine-aspartic acid) sequence (Dennis M S, etc., Proc Natl Acad Sci 1990; 87:2471-2475). The sequences containing RGD have integrin antagonist effect, and can reduce the expression of cell surface adhesion molecules, regulate the intracellular signal transduction, so it has very broad application prospects in tumor treatment.

Physiochemical modification is an important process to enhance the effectiveness of polypeptide(s) or proteins in the treatment or biotechnology. When the modified substances are bound to the protein or polypeptide(s) in appropriate ways or the protein polypeptide(s) are modified, they can modify many features, while the main biological activity functions, such as the enzyme activity or specific binding sites, can be retained. Physiochemical modification process can enhance the drug properties through the following means, firstly, the modified substances can be bound to the surface of proteins or polypeptide(s) to enhance the molecular size, to carry large amount of water molecules, and such modified substance-protein is thus increased by 5-10 times; secondly, the physiochemical modification can not only dissolve the previously insoluble proteins, but also has the feature of high degree of mobility; in addition, the modification on the protein polypeptide s can reduce the filtration of drugs through kidney, and reduce its pyrogen property, but also can reduce the digestion of protease, and enhance its transport through the protection molecules by preventing from human body's immune system attacks; meanwhile, because it avoids the human body's defense mechanism, it stays on the action site much longer, and thus enhancing the drug concentration of local parts. The PEG-polypeptide(s) (or PEG-protein) products currently available on the markets include many varieties, such as the new drug SD/01 developed by the world's largest biotechnology company Amgen, which is the PEG-modified granulocyte colony-stimulating factor G-CSF, and the long-acting G-CSF of the early product of Amgen; while the sales of G-CSF in 1999 was 1.22 billion USD, and 1.26 billion USD in 2000, so the market of modified protein polypeptide(s) products is very huge.

### SUMMARY

Until so far, some small peptides encoding antiangiogenesis agent have the effect of inhibiting angiogenesis and anti-tumors. In this study, different sequences containing arginine-glycine-aspartic amino acid are added to both ends of the small polypeptides that inhibit angiogenesis to construct a kind of antiangiogenesis agent having binding effect and affinity with the integrin.

A first embodiment may provide a kind of highly efficient antiangiogenesis agent having binding effect and affinity with the integrin. The highly efficient antiangiogenesis agent can be synthesized. A second embodiment may be to provide a production method of said “highly efficient antiangiogenesis agent having binding effect and affinity with the integrin”. The target genes can be synthesized or the product can be obtained by cloning of the target genes into the prokaryotic expression vector or a eukaryotic expression vector by PCR amplification. A third embodiment may be to provide the physiochemical modification methods of said “highly efficient antiangiogenesis agent having binding effect and affinity with the integrin”. Many modification methods are applied to modification of angiogenesis inhibition polypeptides, including polyethyleneglycol modification, heparin modification, dextran modification, polyvinylpyrrolidone modification, polyethyleneglycol-poly-amino acid copolymer modification, palmitic acid modification, poly-sialic acid modification, and liposomes and nano-technology modification, to obtain a variety of modified products. A fourth embodiment may be to provide the uses of said “highly efficient antiangiogenesis agent having binding effect and affinity with the integrin” and its modified products, i.e. application of highly efficient antiangiogenesis agent having binding effect and affinity with the integrin and its modified products in the production of mendicants for treating tumors.

To achieve the above first embodiment:

At both ends of angiogenesis inhibitor polypeptide Ile-Val-Arg-Arg-Ala-Asp-Arg-Ala-Ala-Val-Pro (known as ES-2), at least one end is connected with polypeptides with binding effect and affinity with the integrin family, and said “polypeptides with binding effect and affinity with the integrin family” refer to the sequences containing Arg-Gly-Asp or Arg-Gly-Asp-linker, and said linker and all linker refer to one or more different amino acids.

Said polypeptide sequences containing Arg-Gly-Asp or Arg-Gly-Asp-linker are selected from the following:

Arg-Gly-Asp-linker -Ile-Val-Arg-Arg-Ala-Asp-Arg-
Ala-Ala-Val-Pro(P1),
Arg-Gly-Asp-Ile-Val-Arg-Arg-Ala-Asp-Arg-Ala-Ala-
Val-Pro(P2),
Ile-Val-Arg-Arg-Ala-Asp-Arg-Ala-Ala-Val-Pro-
linker-Arg-Gly-Asp(P3),
Ile-Val-Arg-Arg-Ala-Asp-Arg-Ala-Ala-Val-Pro-Arg-
Gly-Asp(P4),
Arg-Gly-Asp-Ile-Val-Arg-Arg-Ala-Asp-Arg-Ala-Ala-
Val-Pro-Arg-Gly-Asp(P5),
Arg-Gly-Asp-linker-Ile-Val-Arg-Arg-Ala-Asp-Arg-
Ala-Ala-Val-Pro-linker-Arg-Gly-Asp(P6),
Arg-Gly-Asp-Ile-Val-Arg-Arg-Ala-Asp-Arg-Ala-Ala-
Val-Pro-linker-Arg-Gly-Asp(P7),
or
Arg-Gly-Asp-linker-Ile-Val-Arg-Arg-Ala-Asp-Arg-
Ala-Ala-Val-Pro-Arg-Gly-Asp(P8).

For further modification of the above technical arts, said polypeptide sequences containing Arg-Gly-Asp or Arg-Gly-Asp-linker can use the sequence of Ala-Cys-Asp-Cys-Arg-Gly-Asp-Cys-Phe-Cys or Ala-Cys-Asp-Cys-Arg-Gly-Asp-Cys-Phe-Cys-linker.

Said sequences containing Ala-Cys-Asp-Cys-Arg-Gly-Asp-Cys-Phe-Cys or Ala-Cys-Asp-Cys-Arg-Gly-Asp-Cys-Phe-Cys-linker are selected from the following:

Ala-Cys-Asp-Cys-Arg-Gly-Asp-Cys-Phe-Cys-linker-
Ile-Val-Arg-Arg-Ala-Asp-Arg-Ala-Ala-Val-Pro(P9),
Ala-Cys-Asp-Cys-Arg-Gly-Asp-Cys-Phe-Cys-Ile-Val-
Arg-Arg-Ala-Asp-Arg-Ala-Ala-Val-Pro(P10), Ile-
Val-Arg-Arg-Ala-Asp-Arg-Ala-Ala-Val-Pro-linker-
Ala-Cys-Asp-Cys-Arg-Gly-Asp-Cys-Phe-Cys(P11),Ile-
Val-Arg-Arg-Ala-Asp-Arg-Ala-Ala-Val-Pro-Ala-Cys-
Asp-Cys-Arg-Gly-Asp-Cys-Phe-Cys(P12), Ala-Cys-Asp-
Cys-Arg-Gly-Asp-Cys-Phe-Cys-Ile-Val-Arg-Arg-Ala-
Asp-Arg-Ala-Ala-Val-Pro-Ala-Cys-Asp-Cys-Arg-Gly-
Asp-Cys-Phe-Cys(P13), Ala-Cys-Asp-Cys-Arg-Gly-Asp-
Cys-Phe-Cys-linker-Ile-Val-Arg-Arg-Ala-Asp-Arg-
Ala-Ala-Val-Pro-Ala-Cys-Asp-Cys-Arg-Gly-Asp-Cys-
Phe-Cys(P14), Ala-Cys-Asp-Cys-Arg-Gly-Asp-Cys-Phe-
Cys-Ile-Val-Arg-Arg-Ala-Asp-Arg-Ala-Ala-Val-Pro-
linker-Ala-Cys-Asp-Cys-Arg-Gly-Asp-Cys-Phe-Cys
(P15),Ala-Cys-Asp-Cys-Arg-Gly-Asp-Cys-Phe-Cys-
linker-Ile-Val-Arg-Arg-Ala-Asp-Arg-Ala-Ala-Val-Pro-
linker-Ala-Cys-Asp-Cys-Arg-Gly-Asp-Cys-Phe-
Cys(P16).

Such type of connection can enhance the targeting of angiogenesis inhibition polypeptide Ile-Val-Arg-Arg-Ala-Asp-Arg-Ala-Ala-Val-Pro of (see Table 1).

The two types can be further modified, for example:

Option 1: said polypeptide sequences and base sequences encoding this polypeptide sequence can be formed through chemical synthesis method.
Option 2: said polypeptide sequences and base sequences encoding this polypeptide sequence can be formed through one of the following:
2-1 Synthesize the angiogenesis inhibition genes containing Arg-Gly-Asp integrin-binding peptide segment sequence, and use this sequence as template to design the upstream and downstream primers, and supplement the appropriate cloning restriction sites on the 5′ end and 3′ end, to obtain RGD-ED gene by PCR amplification. The genes are cloned in the vector to screen the positive clones and then carry out nucleotide sequence analysis and identification.
2-2 Said polypeptide sequence and base sequence encoding this polypeptide sequence are formed by the following genetic engineering methods:
RGD-ED gene and recombinant prokaryotic expression vector form the expression plasmid to transform into E. coli, IPTG-induced expression of RGD-ED and the expression products exist in the form of inclusion body.
2-3 Said polypeptide sequences and base sequences encoding this polypeptide sequence are formed through the following genetic engineering:
Carry out inclusion body protein separation, dissolution and renaturation, and conduct ion-exchange chromatography for separation and purification of RGD-ED protein products and collect filtration liquid, and then frozen-drying.
2-4. Said polypeptide sequences and base sequences encoding this polypeptide sequence are formed through the following genetic engineering:
Recombination of all RGD-ED genes and eukaryotic expression vector forms the expression plasmid and transform eukaryotic cells, to induce the expression of RGD-ED, and then the expression products are isolated and purified.
Option 3: said polypeptide sequences and base sequences encoding this polypeptide sequence can be modified to enhance its in vivo half-life and enhance its targeting;
3. Said polypeptides having affinity and binding capacity of integrin family are the polypeptide products after modified by polyethyleneglycol or heparin, or dextran, or polyvinylpyrrolidone, or polyethyleneglycol-poly-amino acid copolymer, or palmitic acid or polysialic acid or liposomes, or by nanotechnology.

To achieve the second object of the invention—the production method of an angiogenesis inhibitor having affinity and binding capability of integrin, the steps are as follows:

At both ends of angiogenesis inhibitor polypeptide Ile-Val-Arg-Arg-Ala-Asp-Arg-Ala-Ala-Val-Pro, at least one end is connected with polypeptide s with binding effect and affinity with the integrin family, and said “polypeptide s with binding effect and affinity with the integrin family” refer to the sequences containing Arg-Gly-Asp or Arg-Gly-Asp-linker, or Ala-Cys-Asp-Cys-Arg-Gly-Asp-Cys-Phe-Cys or Ala-Cys-Asp-Cys-Arg-Gly-Asp-Cys-Phe-Cys-linker, of which, -linker refers to one or more different amino acids.

For further restriction of the above production methods, there are the following specific production methods:

1. Said polypeptide sequences and base sequences encoding this polypeptide sequence are formed by chemical synthesis including solid phase and liquid phase methods.
2. Said polypeptide sequences and base sequences encoding this polypeptide sequence are formed through one of the following genetic engineering methods:
2-1. Synthesize the angiogenesis inhibitor gene sequence containing Arg-Gly-Asp integrin-binding sequence polypeptide segment, and use this sequence as template to design the upstream and downstream primers, and supplement the appropriate cloning restriction sites on the 5′ end and 3′ end, to obtain RGD-ED gene by PCR amplification. The genes are cloned in the vector to screen the positive clones and then carry out nucleotide sequence analysis and identification.
2-2. Said polypeptide sequences and base sequences encoding this polypeptide sequence are formed by the following genetic engineering methods:
RGD-ED gene and recombinant prokaryotic expression vector form the expression plasmid to transform into E. coli, IPTG-induced expression of RGD-ED.
2-3. Said polypeptide sequences and base sequences encoding this polypeptide sequence are formed through the following genetic engineering:
Carry out inclusion body protein separation, dissolution and renaturation, and conduct ion-exchange chromatography for separation and purification of RGD-ED protein products and collect filtration liquid, and then frozen-drying.
2-4. Said polypeptide sequences and base sequences encoding this polypeptide sequence are formed through the following genetic engineering:
Recombination of all RGD-ED genes and eukaryotic expression vector forms the expression plasmid and transform eukaryotic cells, to induce the expression of RGD-ED, and then the expression products are isolated and purified.

To achieve the third embodiment—a method to modify the polypeptide sequences and base sequences encoding this polypeptide sequence, the steps are as follows:

1. To implement polyethylene glycol (PEG) modification of all polypeptide sequences of this invention, with linear PEG (relative molecular weight of 2000˜30000 Dα) or branched PEG (relative molecular weight of 40000˜60000 Dα), including: (1) PEG-Vinylsulphone; (2) PEG-Iodoacetamide; (3) PEG-Maleimide; (4) PEG-Orthopyridyldisulfide; (5) SC-mPEG or SS-PEG, or PEG-isocyanate (7)(8) (6) mPEG-ALD.
2. Implement heparin modification of all polypeptide sequences in the invention.
3. Implement dextran modification of all polypeptide sequences in the invention.
4. Implement polyvinylpyrrolidone (PVP) modification of all polypeptide sequences in the invention.
5. Implement polyethylene glycol-poly-amino acid copolymer modification of all polypeptide sequences in the invention.
6. Implement palmitic acid modification of all polypeptide sequences in the invention.
7. Implement colominic acid modification of all polypeptide sequences in the invention.
8. Implement liposomes modification of all polypeptide sequences in the invention, including REV, DRV and Mvl.
9. Implement nano-technology modification of all polypeptide sequences in the invention.

The fourth embodiment and the highly efficient angiogenesis inhibitor with affinity or binding ability to Integrin in the production of medicaments for treating tumors may be achieved.

Said applications in the production of medicaments for treating tumors include the production of nano-drugs of all polypeptide sequences in the invention, including the production of polylactic acid (PLA) nano-particles or micro-balls, or poly-butylcyanoacrylate (PBCA) nano-particles or micro-balls, or chitosan nano-particles or micro-balls.

The endothelial cell proliferation assay, CAM analysis and in vivo anti-tumor test in mice were carried out to analyze the conditions of binding with tumor cells. These tests showed that the products in the present invention can significantly enhance and improve the effect of angiogenesis inhibitors in inhibiting endothelial cell growth and anti-tumor, with low use amount, reducing the costs, thus, the highly efficient angiogenesis inhibitor in the present invention is scientific, reasonable, feasible and effective, can be used as the medicaments for treating tumors.

The present invention can achieve the purposes as follows:

Said highly efficient angiogenesis inhibition polypeptide s and its physiochemical modified products can be used to prepare the medicaments for treating the human angiogenesis-related diseases-tumors.

Compared with the similar type of products, the resulting products have highly efficient and specific inhibition on the endothelial cell proliferation and anti-tumor effects, with small dosage, reducing the side effect of medicament treatment.

In the present invention, modifications of polypeptide s are carried out, which extend the half-life of polypeptide s (T1/2), enhance the stability, reduce the immunogenicity and antigenicity, change the molecular structure and thus improve the medicament kinetic and pharmacodynatics nature, enhance the blood concentration of the affecting parts. Meanwhile, the modified polypeptide s present better tolerance compared with the non-modified polypeptide s, and enhance the clinical application scope and efficacy in the future.

### BRIEF DESCRIPTION OF THE DRAWINGS

FIG. 1 Results of HPLC purified RGD-ED analysis

FIG. 2 CAM analysis of RGD-ED inhibiting angiogenesis: A, blank control, B, C, D represent the treatment groups of 0.05 μg, 0.1 μg and 0.2 μg respectively

FIG. 3 RGD-ED in vivo tumor suppression effect

FIG. 4 in vivo tumor suppression effect of polyethylene glycol (PEG) and liposome-modified polypeptide s: 1, 2, 3 represent the effect of inhibition of human hepatocellular carcinoma of non-modified RGD-ED and liposome-modified and PEG-modified RGD-EDs respectively

### DETAILED DESCRIPTION

### 1. RGD-ED Gene Cloning and Construction of Prokaryotic Expression Vector

The bases encoding RGD-ED polypeptide sequences were synthesized as the template; and the upstream primer and downstream primer were synthesized, of which, the upstream primer was added with NdeI restriction site; while the downstream primers contained Arg-Gly-Asp sequence and XhoI site, then PCR amplification was carried out, and the amplification products were recovered through agarose gel electrophoresis and purified, then NdeI and XhoI digestion, and then cloned into prokaryotic expression vector pw, PCR screening of positive clones, and nucleotide sequence analysis confirmed that the sequence mutations have occurred in the design.

Synthetic primer 1:
5′GGAATTCCATATG ATCGTGCGCCGTGCCGACCGC3′
Synthetic primer 2:
5′CCGCTCGAGGCAGAAGCAGTCACCACGGCA3′

of which, primer 1 encoding NdeI site and part of Ile-Val-Arg-Arg-Ala-Asp-Arg-Ala-Ala-Val-Pro sequence, and the primer 2 encoding XhoI site and genes containing Arg-Gly-Asp sequences.

2. To compare the actual effect of the designed angiogenesis inhibitor RGD-ED in the present invention, in this embodiment, we entrusted the company to synthesize the polypeptide Ile-Val-Arg-Arg-Ala-Asp-Arg-Ala-Ala-Val-Pro (ED) not containing Arg-Gly-Asp sequence.

### 3. Induced Expression of Recombinant Bacteria

The expression plasmid was used to transfected the E. coli, after the recombinant bacteria was induced to expression for 3 h by 1 mMIPTG. The cells were collected and broken under ultrasound wave, and the supernatant and precipitation were centrifuged and separated, and then subject to 15% SDS-PAGE electrophoresis analysis. The SDS-PAGE stained by Coomassie brilliant blue was scanned (UVP White/Ultraviolet transilluminator) and the expression results were analyzed.

### 4. Inclusion Body Separation, Dissolution and Renaturation

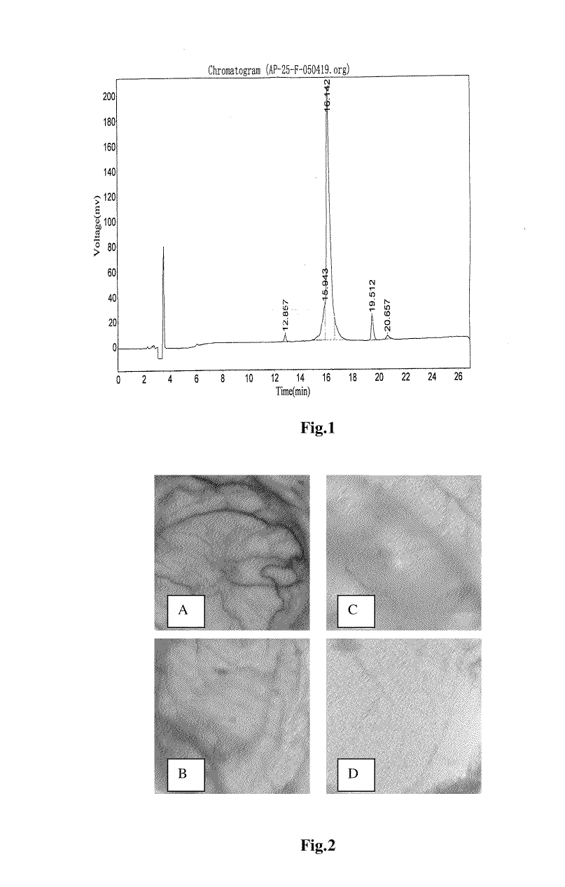

The bacteria were broken by ultrasound wave and separated through centrifugation, then the inclusion body precipitation was washed with 0.1 Mtris and sodium deoxycholate. The precipitation was dissolved in the sodium Lauryl sarcosine (SLS), centrifugation for 5 min at 10000 rpm under 4° C. The supernatant was dialyzed at 4° C. with the dialysis solutions of buffer A (10 mM Tris-HCl, 0.1 mM oxidized glutathione and 1 mM reduced glutathione, pH7.4), replaced for 3 times in total, and then dialysis for one time with the dialysis solution of buffer B (10 mM Tris-HCl, pH7.4). The samples were directly SP-Sepharose Fast Flow (Amersham Pharmacia Biotech) chromatography. Buffer B solution was washed with 0.6M NaCl, Tris-HCl, pH7.4, and 1M NaCl, Tris-HCl, pH7.4, then elution step by step was carried out and the elution solution was mixed. The buffer B solution was concentrated and freeze-dried after dialysis. The chromatography results were shown in FIG. 1.

### 5. Analysis of Endothelial Cell Proliferation

The culture of BCE cells and NIH 3T3 cells, method: the culture solution DMEM contained 10% inactivated calf serum (BCS), 1% antibiotics and 3 ng/ml bFGF. Cell proliferation analysis method is as follows: washed the cells with PBS, digested with trypsin, added with culture solution suspension cells and centrifuged and collected cells, and regulated the cell concentrations to 25,000 cells/ml. The cells were moved to 6-well plates (0.5 ml/well) and cultured for 24 h. Replaced the culture medium as 1 ml DMEM, 5% BCS, 1% antibiotics, 1 ng/ml bFGF. Different doses of samples were added to each hole, and further cultured for 48 h, digested the cells with trypsase, re-suspended in PBS, fixed with 70% ice-cold ethanol and stained with 7-AAD, and then conducted analysis with flow cytometry.

The results showed that: Recombinant RGD-ED can specifically inhibit the endothelial cell-BCE cell proliferation, but have no inhibitory effect on non—NIH 3T3 endothelial cells. The ED₅₀ of inhibition of BCE proliferation was about 0.1 μg/ml, but the polypeptide ED₅₀ without RGD sequence was about 0.8 μg/ml, and the ED₅₀ of endostatin was about 0.5 μg/ml. The above tests showed that, the highly-efficient angiogenesis inhibitors actually enhanced the bioactivity of the existing angiogenesis.

### 6. CAM Analysis

To detect the in vivo anti-angiogenic activity, CAM analysis was carried out. All the tests were carried out on the ultra-clean platform under sterile conditions. The 6-day disinfected chick embryos were cultured under the condition of 37° C., 90% humidity. After 2 days, the top of each egg was punched and the reagents were dripped into the sterile Whatman filter paper, then put on the CAMs vascular clustered area, cultivated for 48 hours, and observed the chick embryos and CAMs and took pictures.

As shown in FIG. 2, to evaluate the RGD-ED in vivo anti-angiogenesis activity, different doses of RGD-ED were used to carry out CAM test, of which, 0.05 μg, 0.1 μg and 0.2 μg of the RGD-ED were able to significantly inhibit the neovascularization and angiogenesis, while 0.5 μgRGD-ED could completely inhibit the angiogenesis and cause chick embryo death. RGD-ED had a potential role in inhibiting angiogenesis.

### 7. Polypeptide(s) of this Invention can be Artificially Synthesized with Solid or Liquid Phase Methods

### 8. PEG Modification of Polypeptide

### 8.1. N-Terminal Amino Acylation Modification

The polypeptide with weight percentage of about 1-70%, preferably about 20-50% was mixed with about 20%-95% of the polyethylene glycol SC-mPEG (with an average molecular weight of 5000) or SS-PEG (succinamide-type) or PEG-isocyanate solution, preferably about 50-93% of the above-mentioned modified substance, mixed fully and shaken on the shaker at temperature of 4° C.-40° C., preferably 25° C.-37° C. for more than 10 min, and then the N-terminal modified products were separated through ion-exchange chromatography.

### 8.2. Carboxy-Terminal Modification

The polypeptides with weight percentage of about 1-70%, preferably about 20-50% were fully mixed with about 20%-95% mPEG-NH2 (average molecular weight of 5000) and small amount of DCCI (dicyclohexyl carbodiimide) solution, preferably about 50-93% of the above modified substances, and then shaken at the shaker at 22° C.-37° C. preferably 4° C.-40° C. for more than 10 min, preferably 90 min. The carboxyl group could bind with the amino-group of mPEG-NH2 to form amide bond, and then N-terminal modified product was obtained through RP-HPLC separation.

### 8.3. Thiol-Terminal Modification

The polypeptides with weight percentage of about 1-70%, preferably about 1-30% were fully mixed with about 20%-95% mPEG-MAL (average molecular weight of 5000) solution. Preferably about 70-94% of the above modified substance was fully mixed even, and shaken on the shaker at 4° C.-40° C., preferably 20° C.-37° C. for more than 10 min, preferably 10 min, and then N-terminal modified products were obtained through ion-exchange separation.

### 9. Other Modification Methods

Other modification methods refer to PEG modification, of which, the PEG-modification and liposome-modification have the best modification effects (see FIG. 4).

### 10. In Vivo Anti-Tumor Test

The cultured B16F10 melanoma cells were treated with 0.05% trypsin, centrifuged at 1000 rpm for 5 min, suspended again in PBS, inoculated subcutaneously with 0.1 ml of 5×10⁵ cells at the body sides of C57BL/6 mice (6-8 weeks). When the average tumor size reached 200 mm3-300 mm3, the mice were randomly divided into two groups, 7 mice for each group, of which, one group of mice were treated with RGD-ED, and the other group were treated with ED not containing RGD, with the doses of 5 mg/kg/d for the two groups, and the control group of mice were subject to PBS injection. The treatment method was contralateral subcutaneous injection of the inoculated tumors. Every day, the tumor size was measured with a vernier caliper to calculate the tumor volume with the formula: tumor volume=length × width²×0.52, and the treatment efficacy was represented with the tumor inhibition rate within the given period of time: (1−T/C)×100%, T=tumor volume of treatment group, C=tumor volume of control group.

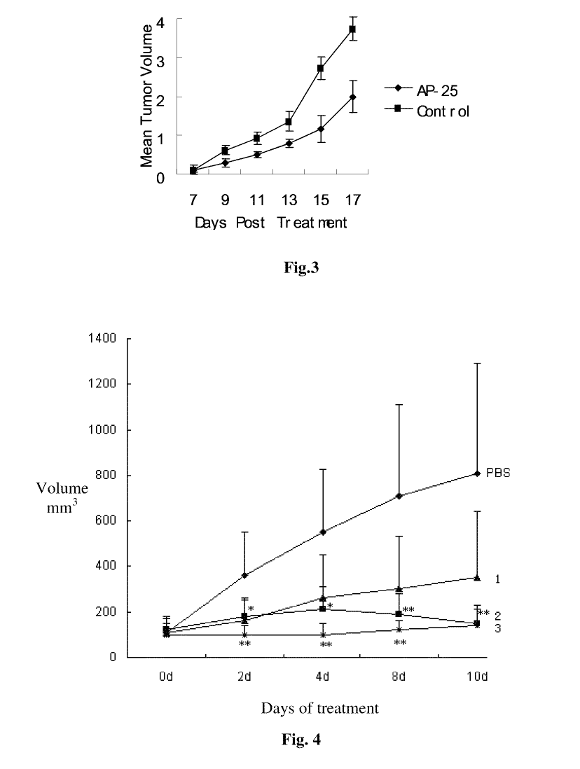

As shown in FIG. 3, the results showed that, on the 9^{th} day, the RGD-ED tumor inhibition rate was 58%, while the tumor inhibition rate of polypeptide ED not containing RGD sequence was 28%. The above tests showed that, the highly efficient angiogenesis inhibitors of this invention could significantly inhibit the tumor growth in the mouse body.

The cultured human liver cancer SGC7901 was treated with 0.05% trypsin, centrifuged at 1000 rpm for 5 min, suspended again in PBS, inoculated subcutaneously with 0.1 ml of 5×10⁵ cells on the body sides of nude mice (6-8 weeks). When the average tumor size reached 100 mm³-200 mm³, the mice were randomly divided into three groups, 7 mice for each group, of which, one group of mice were treated with RGD-ED, one group were treated with RGD-ED modified with liposome, and the third group were treated with RGD-ED modified with polyethylene glycol, with the doses of 3 mg/kg/d for the three groups, and the control group of mice were subject to PBS injection. The treatment method was contralateral subcutaneous injection of the inoculated tumors. Every day, the tumor size was measured with a vernier caliper to calculate the tumor volume according to the formula: tumor volume=length × width²×0.52, and the treatment efficacy was represented by the tumor inhibition rate within the given period of time: (1−T/C)×100%, T=tumor volume of treatment group, C=tumor volume of control group.

As shown in FIG. 4. the results showed that, on the 10^{th} day, the RGD-ED tumor inhibition rate was 68%, the liposome-modified RGD-ED tumor inhibition rate was 72%, and the polyethylene glycol-modified RGD-ED tumor inhibition rate was 78%. The above test showed that, the modification products obtained according to the designed modification method could significantly inhibit the tumor growth in the mouse body.

### Example 2

The procedure was carried out basically as Example 1, but, wherein the gene sequence:

Arg-Gly-Asp containing Primer 2 encoding XhoI site should be replaced by the sequence Arg-Gly-Asp-Gly-Gly-Gly-Gly, and then the amplified Ile-Val-Arg-Arg-Ala-Asp-Arg-Ala-Ala-Val-Pro-Gly-Gly-Gly-Gly-Arg-Gly-Asp sequence was cloned.

### Example 3

The procedure was carried out basically as Example 1, but, the full-length gene synthesis of gene sequence of Arg-Gly-Asp-Gly-Gly-Gly-Gly-Ile-Val-Arg-Arg-Ala-Asp-Arg-Ala-Ala-Val-Pro, and then cloned.

### Example 4

The procedure was carried out basically as Example 1, but, the full-length gene synthesis of gene sequence of Arg-Gly-Asp-Ile-Val-Arg-Arg-Ala-Asp-Arg-Ala-Ala-Val-Pro, and then cloned.

### Example 5

The procedure was carried out basically as Example 1, but, the full-length gene synthesis of gene sequence of Arg-Gly-Asp-Ile-Val-Arg-Arg-Ala-Asp-Arg-Ala-Ala-Val-Pro-Arg-Gly-Asp, and then cloned.

### Example 6

The procedure was carried out basically as Example 1, but, the full-length gene synthesis of gene sequence of Arg-Gly-Asp-Gly-Gly-Gly-Gly-Ile-Val-Arg-Arg-Ala-Asp-Arg-Ala-Ala-Val-Pro-Gly-Gly-Gly-Gly-Arg-Gly-Asp, and then cloned.

### Example 7

The procedure was carried out basically as Example 1, but, the full-length gene synthesis of gene sequence of Arg-Gly-Asp-Ile-Val-Arg-Arg-Ala-Asp-Arg-Ala-Ala-Val-Pro-Gly-Gly-Gly-Gly-Arg-Gly-Asp, and then cloned.

### Example 8

The procedure was carried out basically as Example 1, but, the full-length gene synthesis of gene sequence of Arg-Gly-Asp-Gly-Gly-Gly-Gly-Ile-Val-Arg-Arg-Ala-Asp-Arg-Ala-Ala-Val-Pro-Arg-Gly-Asp, and then cloned.

### Example 9

The procedure was carried out basically as Example 1, but, the full-length gene synthesis of gene sequence of Ala-Cys-Asp-Cys-Arg-Gly-Asp-Cys-Phe-Cys-Gly-Gly-Gly-Gly-Ile-Val-Arg-Arg-Ala-Asp-Arg-Ala-Ala-Val-Pro, and then cloned.

### Example 10

The procedure was carried out basically as Example 1, but, the full-length gene synthesis of gene sequence of Ala-Cys-Asp-Cys-Arg-Gly-Asp-Cys-Phe-Cys-Ile-Val-Arg-Arg-Ala-Asp-Arg-Ala-Ala-Val-Pro, and then cloned.

### Example 11

The procedure was carried out basically as Example 1, but, the full-length gene synthesis of gene sequence of Ile-Val-Arg-Arg-Ala-Asp-Arg-Ala-Ala-Val-Pro-Gly-Gly-Gly-Gly-Ala-Cys-Asp-Cys-Arg-Gly-Asp-Cys-Phe-Cys, and then cloned.

### Example 12

The procedure was carried out basically as Example 1, but, the full-length gene synthesis of gene sequence of Ile-Val-Arg-Arg-Ala-Asp-Arg-Ala-Ala-Val-Pro-Ala-Cys-Asp-Cys-Arg-Gly-Asp-Cys-Phe-Cys, and then cloned.

### Example 13

### PEG Modification of Polypeptide s

### 13.1. N-Terminal Amino Acylation Modification

Polypeptides with weight percentage of about 1-70%, preferably about 20-50% was mixed with about 20%-95% of polyethylene glycol SC-mPEG (with an average molecular weight of 5000) or SS-PEG (succinamides) or PEG-isocyanate solution, preferably about 50-93% of the above-mentioned modified substance, then fully mixed even, and shaken on a shaker at temperature of 4° C.-40° C., preferably 25° C.-37° C. for more than 10 min, and then the N-terminal modified products were separated through ion-exchange chromatography.

### 13.2. Carboxy-Terminal Modification

Polypeptides with weight percentage of about 1-70%, preferably about 20-50% were fully mixed with about 20%-95% mPEG-NH2 (average molecular weight of 5000) and small amount of DCCI (dicyclohexyl carbodiimide) solution, preferably about 50-93% of the above modified substances were fully mixed even, and then shaken at a shaker at 4° C.-40° C., preferably 22° C.-37° C. for more than 10 min, preferably 90 min The carboxyl group could bind with the amino-group of mPEG-NH2 to form amide bond, and then N-terminal modified products were obtained through RP-HPLC separation.

### 13.3. Thiol-Terminal Modification

Polypeptides with weight percentage of about 1-70%, preferably about 1-30% were fully mixed with about 20%-95% mPEG-MAL (average molecular weight of 5000) solution. Preferably about 70-94% of the above modified substance was fully mixed even, and shaken on a shaker at 4° C.-40° C., preferably 20° C.-37° C. for more than 10 min, preferably 60 min, and then N-terminal modified products were obtained through ion-exchange separation.

### 13.4. In Vivo Anti-Tumor Test

The cultured human liver cancer SGC7901 was treated with 0.05% trypsin, centrifuged at 1000 rpm for 5 min, suspended again in PBS, inoculated subcutaneously with 0.1 ml of 5×10⁵ cells on the body sides of nude mice (6-8 weeks). When the average tumor size reached 100 mm³-200 mm³, the mice were randomly divided into three groups, 7 mice for each group, of which, one group of mice were treated with RGD-ED, one group were treated with liposome-modified RGD-ED, and the third group were treated with polyethylene glycol-modified RGD-ED, with the doses of 3 mg/kg/d for the three groups, and the control group of mice were subject to PBS injection. The treatment method was contralateral subcutaneous injection of the inoculated tumors. Every day, the tumor size was measured with a vernier caliper to calculate the tumor volume according to the formula: tumor volume=length × width²×0.52, and the treatment efficacy was represented by the tumor inhibition rate within the given period of time: (1−T/C)×100%, T=tumor volume of treatment group, C=tumor volume of control group. As shown in FIG. 4, the results showed that, on the 10^{th} day, the RGD-ED tumor inhibition rate was 68%, the liposome-modified RGD-ED tumor inhibition rate was 72%, and the polyethylene glycol-modified RGD-ED tumor inhibition rate was 78%. The above test showed that, the modification products obtained according to the designed modification method could significantly inhibit the growth of tumors in the mouse body.

### Example 14

The procedure was carried out basically as Example 13, but, wherein heparin modification was adopted.

Polypeptides with weight percentage of about 10%-90%, preferably about 25%-50% were mixed with about 10%-90% activated low molecular weight of heparin, preferably about 43%-75%, and slightly shaken in buffer solution of pH 7-9 at 4° C. for more than 18 h, and then the free amino-modified products were separated through ion-exchange chromatography method.

### Example 15

The procedure was carried out basically as Example 13, but, wherein PEG-PLA modification was adopted.

80 mg-120 mg of PEG-PLA and 15 mg-30 mg polypeptide s were dissolved in 40 ml of dimethylformamide (DMF) and the resulting mixture was transferred to dialysis bag with MWCO of 3500, and dialyzed for 48 h in 3 L-4 L of distilled water, removed of precipitation through centrifugation. The supernatant was filtered through 0.45 nm filter membrane to obtain the polymer micellar solution.

### Example 16

The procedure was carried out basically as Example 13, but, wherein dextran modification was adopted.

1 g dextran (molecular weight of 35,000) was activated and added with 10 mg-60 mg of polypeptide, preferably 20 mg-45 mg, slowly shaken on a shaker at 4° C. for more than 15 h, and then the free amino-modified products were separated through ion-exchange chromatography method.

### Example 17

The procedure was carried out basically as Example 13, but, wherein palmitic acid modification was adopted.

Polypeptides with weight percentage of about 5%-90%, preferably about 25%-60% were mixed with about 10%-95% activated palmitic acid, preferably about 43%-90%, and shaken in a shaker at 25-° C.-37° C. for reactions for more than 30 min, and then the free amino-modified products were separated through ion-exchange chromatography method.

### Example 18

The procedure was carried out basically as Example 13, but, wherein colominic acid modification was adopted.

Polypeptides with weight percentage of about 1%-90%, preferably about 7%-50% were mixed with about 10%-95% activated colominic acid, preferably about 50%-93%, and shaken in a shaker at 4° C.-40° C. preferably 25-° C.-37° C. for reactions for more than 15 h, and then the free amino-modified products were separated through ion-exchange chromatography method.

### Example 19

The procedure was carried out basically as Example 13, but, wherein liposomes modification was adopted, including REV, DRV and Mvl.

A certain percentage of soybean lecithin and cholesterol were dissolved in chloroform, and evaporated into thin film at 35° C.-45° C. under reduced pressure condition, and then dissolved in anhydrous ether. 6 mg-10 mg polypeptide s were weighed and dissolved in 6 ml pH6-8 phosphate buffer. The buffer solution was mixed with the phospholipid solution, shaken for 4 min-9 min under ultrasonic condition, and then evaporated to remove organic solvent at 20° C.-30° C. under reduced pressure condition, and then dried under vacuum conditions at 60° C., and then immersed with phosphate buffer solution for 4 h to obtain the liposome suspension. The suspension was circulated for several times through a high-pressure homogenization machine to obtain the liposomes.

### Example 20

The procedure was carried out basically as Example 13, but, wherein nano-drug preparation was adopted, including PLA, PBCA and chitosan nanoparticles.

10 mg-50 mg of chitosan was mixed with 20 ml-40 ml of water, and under ultrasonic condition for 20-40 min, to obtain micro-sphere dispersions. The added 3.5 mg-6 mg polypeptide s were dissolved in 2 ml-5 ml of anhydrous methanol, then slowly dripped with micro-sphere dispersion during the ultrasonic process to encapsulate the polypeptide s, and then centrifuged to obtain nano-particles.

### Example 21

Analysis of the binding of polypeptide s and integrin by flow cytometry. Bel-7402 tumor cells were used, which can express integrin. The cells were cultured in a 24-well plate, and the cells were collected and washed with ice-cold PBS twice. Before labeling, the cells were suspended in PBS +1% BSA, maintained for 30 min, added with 1 μl FITC labeled polypeptide s (P1, P2, P3, P4, P5, P6, P7, P8, P9, P10, P11, P12, P13, P14, P15, P16, RGD and ES-2) for reaction for 25 min. After labeling, the cells were collected and washed twice with PBS, suspended again in 400 μl PBS and analysis was conducted with a flow cytometry (Becton Dickinson, USA). FITC fluorescence was determined at FL1 channel The cells with FITC feature would be further analyzed. The results showed that, after modification, adding of polypeptide containing RGD sequences can specifically bind with the tumor cells expressing integrin (Table 1).

TABLE 1
Results of tumor targeting analysis after adding sequences
containing RGD sequences

| | Dosage | |
|---|---|---|
| Group | (μg/μl) | Binding Value |
| Negative control group(ES-2) | 1 | 18.35 |
| P1 | 1 | 63.21 |
| P2 | 1 | 62.15 |
| P3 | 1 | 58.96 |
| P4 | 1 | 60.13 |
| P5 | 1 | 59.40 |
| P6 | 1 | 60.27 |
| P7 | 1 | 60.25 |
| P8 | 1 | 58.30 |
| P9 | 1 | 66.04 |
| P10 | 1 | 63.28 |
| P11 | 1 | 60.20 |
| P12 | 1 | 67.28 |
| P13 | 1 | 59.25 |
| P14 | 1 | 58.32 |
| P15 | 1 | 64.11 |
| P16 | 1 | 66.12 |
| RGD | 1 | 59.45 |

### Example 22

Survey on the half-life of polypeptides in plasma at 37° C. after modification. Preparation of blood drug samples: after crude drug (volume V) (800 ug/ml) +1V plasma were mixed and diluted with 2V water evenly, incubated for 0, 5, 30 min at 37° C. respectively, immediately heated at 80° C. for 30 min; the negative control: after 1V plasma were diluted and mixed with 3 V water evenly, and heated for 30 min at 80° C. The positive control group: after 1V crude drug (800 ug/ml) were diluted and mixed evenly with 3V water, heated for 30 min at 80° C. The samples were the modified products, heated at 14000 rpm for 10 min. The supernatant was fetched for HPLC analysis with the injection size of 20 ul. The results showed that, the half-life of polypeptides after modification significantly increased (See Table 2).

TABLE 2
Analysis of anti-tumor effect of polypeptides after modification

| | Dosage | Half life of |
|---|---|---|
| Group | (800 μg/ml) | plasma (min) |
| Crude drug P1 | 20 μl | 7 |
| Polyethylene glycol-modified substance | 20 μl | 18 |
| Heparin-modified substance | 20 μl | 16 |
| Dextran-modified substance | 20 μl | 15 |
| polyvinyl pyrrolidone-modified substance | 20 μl | 24 |
| Polyethylene glycol-poly-amino acid | 20 μl | 32 |
| copolymer modified substance | | |
| Palmitic acid-modified substance | 20 μl | 12 |
| Polysialic acid modified substance | 20 μl | 15 |
| Liposome modified substance | 20 μl | 21 |
| Preparation of P1 polypeptide with | 20 μl | 66 |
| nano-particles | | |

1. A highly efficient antiangiogenesis agent, wherein at the both ends of the polypeptide Ile-Val-Arg-Arg-Ala-Asp-Arg-Ala-Ala-Val-Pro of antiangiogenesis agent, at least one end is bound with polypeptides with binding effect and affinity with the integrin family, and said “polypeptides with binding effect and affinity with the integrin family” refer to the sequences containing Arg-Gly-Asp or Arg-Gly-Asp-linker, and said linker (s) in the present invention refer to one or more different amino acids.

2. The highly efficient antiangiogenesis agent according to claim 1, wherein said polypeptide sequences containing Arg-Gly-Asp or Arg-Gly-Asp-linker are selected from the following:
Arg-Gly-Asp-linker-Ile-Val-Arg-Arg-Ala-Asp-Arg-
Ala-Ala-Val-Pro,
Arg-Gly-Asp-Ile-Val-Arg-Arg-Ala-Asp-Arg-Ala-Ala-
Val-Pro,
Ile-Val-Arg-Arg-Ala-Asp-Arg-Ala-Ala-Val-Pro-
linker-Arg-Gly-Asp,
Ile-Val-Arg-Arg-Ala-Asp-Arg-Ala-Ala-Val-Pro-Arg-
Gly-Asp,
Arg-Gly-Asp-Ile-Val-Arg-Arg-Ala-Asp-Arg-Ala-Ala-
Val-Pro-Arg-Gly-Asp,
Arg-Gly-Asp-linker-Ile-Val-Arg-Arg-Ala-Asp-Arg-
Ala-Ala-Val-Pro-linker-Arg-Gly-Asp,
Arg-Gly-Asp-Ile-Val-Arg-Arg-Ala-Asp-Arg-Ala-Ala-
Val-Pro-linker-Arg-Gly-Asp,
or
Arg-Gly-Asp-linker-Ile-Val-Arg-Arg-Ala-Asp-Arg-
Ala-Ala-Val-Pro-Arg-Gly-Asp.

3. The highly efficient antiangiogenesis agent according to claim 1, wherein said sequences containing Arg-Gly-Asp refer to the sequences containing Ala-Cys-Asp-Cys-Arg-Gly-Asp-Cys-Phe-Cys or Ala-Cys-Asp-Cys-Arg-Gly-Asp-Cys-Phe-Cys-linker.

4. The highly efficient antiangiogenesis agent according to claim 3, wherein said sequences containing Ala-Cys-Asp-Cys-Arg-Gly-Asp-Cys-Phe-Cys or Ala-Cys-Asp-Cys-Arg-Gly-Asp-Cys-Phe-Cys-linker are selected from the following polypeptide sequences:
Ala-Cys-Asp-Cys-Arg-Gly-Asp-Cys-Phe-Cys-linker-
Ile-Val-Arg-Arg-Ala-Asp-Arg-Ala-Ala-Val-Pro, Ala-
Cys-Asp-Cys-Arg-Gly-Asp-Cys-Phe-Cys-Ile-Val-Arg-
Arg-Ala-Asp-Arg-Ala-Ala-Val-Pro, Ile-Val-Arg-Arg-
Ala-Asp-Arg-Ala-Ala-Val-Pro-linker-Ala-Cys-Asp-
Cys-Arg-Gly-Asp-Cys-Phe-Cys, Ile-Val-Arg-Arg-Ala-
Asp-Arg-Ala-Ala-Val-Pro-Ala-Cys-Asp-Cys-Arg-Gly-
Asp-Cys-Phe-Cys, Ala-Cys-Asp-Cys-Arg-Gly-Asp-Cys-
Phe-Cys-Ile-Val-Arg-Arg-Ala-Asp-Arg-Ala-Ala-Val-
Pro-Ala-Cys-Asp-Cys-Arg-Gly-Asp-Cys-Phe-Cys, Ala-
Cys-AsP-Cys-Arg-Gly-Asp-Cys-Phe-Cys-linker-Ile-
Val-Arg-Arg-Ala-Asp-Arg-Ala-Ala-Val-Pro-Ala-Cys-
Asp-Cys-Arg-Gly-Asp-Cys-Phe-Cys, Ala-Cys-Asp-Cys-
Arg-Gly-Asp-Cys-Phe-Cys-Ile-Val-Arg-Arg-Ala-Asp-
Arg-Ala-Ala-Val-Pro-linker-Ala-Cys-Asp-Cys-Arg-
Gly-Asp-Cys-Phe-Cys,
or
Ala-Cys-Asp-Cys-Arg-Gly-Asp-Cys-Phe-Cys-linker-
Ile-Val-Arg-Arg-Ala-Asp-Arg-Ala-Ala-Val-Pro-
linker-Ala-Cys-Asp-Cys-Arg-Gly-Asp-Cys-Phe-Cys.

5. The highly efficient antiangiogenesis agent according to claim 2, wherein said polypeptides having affinity and binding capacity of integrin family are the polypeptide products after modified by polyethyleneglycol or heparin, or dextran, or polyvinylpyrrolidone, or polyethyleneglycol-poly-amino acid copolymer, or palmitic acid or polysialic acid or liposomes, or by nanotechnology.

6. A production method of highly efficient antiangiogenesis agent having affinity and binding capacity of integrins as claimed in claim 1, wherein the procedures are as follows:
At both ends of angiogenesis inhibitor polypeptide Ile-Val-Arg-Arg-Ala-Asp-Arg-Ala-Ala-Val-Pro, at least one end is connected with polypeptides with binding effect and affinity with the integrin family, and said “polypeptides with binding effect and affinity with the integrin family” refer to the sequences containing Arg-Gly-Asp or Arg-Gly-Asp-linker or Ala-Cys-Asp-Cys-Arg-Gly-Asp-Cys-Phe-Cys or Ala-Cys-Asp-Cys-Arg-Gly-Asp-Cys-Phe-Cys-linker, and said linker (s) refer to one or more different amino acids.

7. The production method of highly efficient antiangiogenesis agent having affinity and binding capacity of integrins according to claim 6, wherein said polypeptide sequences and base sequences encoding this polypeptide sequence can be formed through chemical synthesis method.

8. The production method of highly efficient antiangiogenesis agent having affinity and binding capacity of integrins according to claim 6, wherein said polypeptide sequences and base sequences encoding this polypeptide sequence can be formed through the following genetic engineering methods:
Synthesize the angiogenesis inhibition gene sequences containing Arg-Gly-Asp integrin-binding sequence polypeptide segments, and use this sequence as a template to design the upstream and downstream primers, and supplement the appropriate cloning restriction sites on the 5′ end and 3′ end, to obtain RGD-ED gene by PCR amplification. The genes are cloned in the vector to screen the positive clones and carry out nucleotide sequence analysis and identification.
Recombination of RGD-ED gene and prokaryotic expression vector forms the expression plasmid to transform into E. coli, IPTG-induced expression of RGD-ED and the expression products exist in the form of inclusion body;
Carry out inclusion body protein separation, dissolution and renaturation, and conduct ion-exchange chromatography for separation and purification of RGD-ED protein products and collect filtration liquid, and then frozen-drying.
Recombination of all RGD-ED genes and eukaryotic expression vector forms the expression plasmid and transform into eukaryotic cells, to induce the expression of RGD-ED, and then the expression products are isolated and purified.

9. A method of modifying the polypeptide sequences and base sequences encoding this polypeptide sequence as claimed in claim 1, wherein one of the following procedures is adopted:
Implement polyethylene glycol (PEG) modification of said polypeptide sequences;
or implement heparin modification of said polypeptide sequences;
or implement dextran modification of said polypeptide sequences;
or implement polyvinylpyrrolidone (PVP) modification of said polypeptide sequences;
or implement polyethylene glycol-poly-amino acid copolymer modification of all polypeptide sequences;
or implement palmitic acid modification of said polypeptide sequences;
or implement colominic acid modification of said polypeptide sequences;
or implement liposomes modification of said polypeptide sequences, and said liposomes including REV, DRV and Mvl;
or implement nano-technology modification of said polypeptide sequences.

10. The method of modification of highly efficient antiangiogenesis agent according to claim 9, wherein said polypeptide modification steps are:
(1) Polypeptides with weight percentage of 1-70% are mixed with 20%-95% polyethylene glycol, or heparin, or dextran, or polyvinylpyrrolidone, or polyethylene glycol-poly-amino acid copolymer, or palmitic acid or poly-sialic acid or liposomes or nanoparticle solutions;
(2) shaken at a shaker for more than 10 min at 4° C.-40° C., preferably at 25° C.-37° C.;
(3) The modified products are separated.

11. The method of modification of highly efficient antiangiogenesis agent according to claim 10, wherein said step (1) the polyethylene glycol is linear with the relative molecular weight of 2000˜30000 Dα, or branched with relative molecular weight of 40000˜60000 Dα, including: (1) PEG-vinyl sulfonic acid; (2) PEG-Iodoacetamide; (3) PEG-Malay amide; (4) PEG-pyridine disulfide; (5) SC-mPEG or SS-PEG (succinamides), or PEG-isocyanate; (6) mPEG-Propionaldehyde.

12. The highly efficient antiangiogenesis agent according to claim 1, wherein the use of said highly efficient anti-angiogenesis agent for manufacturing medicaments for treating tumors.
